(19) United States
(12) Patent Application Publication
(10) Pub. No.: US 2014/0288035 A1
(43) Pub. Date: Sep. 25, 2014

(54) 18-METHYL-6,7-METHYLENE-3-OXO-17-PREGN-4-ENE-21,17B-CARBOLACTONES, PHARMACEUTICAL PREPARATIONS COMPRISING SAID COMPOUNDS AND USE THEREOF IN THE TREATMENT OF ENDOMETRIOSIS

(71) Applicants: Bayer Intellectual Property Gmbh, Monheim (DE); BAYER PHARMA AKTIENGESELLSCHAFT, Berlin (DE)

(72) Inventors: Jan Hübner, Berlin (DE); Rolf Bohlmann, Berlin (DE); Isabella Gashaw, San Francisco, CA (US); Oliver Martin Fischer, Berlin (DE); Joachim Kuhnke, Potsdam (DE); Norbert Gallus, Berlin (DE); Reinhard Nubbemeyer, Berlin (DE); Ildiko Terebesi, Berlin (DE)

(73) Assignees: BAYER PHARMA AKTIENGESELLSCHAFT, Berlin (DE); BAYER INTELLECTUAL PROPERTY GMBH, Monheim (DE)

(21) Appl. No.: 14/356,165

(22) PCT Filed: Nov. 2, 2012
(86) PCT No.: PCT/EP2012/071700

§ 371 Date: May 4, 2014

(30) Foreign Application Priority Data
Nov. 4, 2011 (DE) PCTEP2011069464
Jul. 23, 2012 (DE) 102012212838.7

(51) Int. Cl. C07J 53/00; A61K 31/585

(52) U.S. Cl. CPC C07J 53/007; A61K 31/585
USPC 514/171; 540/15; 514/173

(57) ABSTRACT

The present invention relates to 18-methyl-6,7-methylene-17-pregn-4-ene-21,17β-carbolactones of general formula I

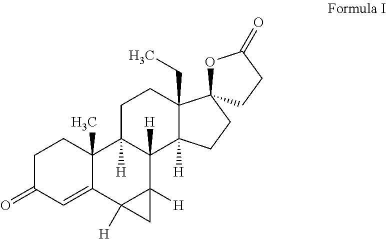

wherein the 6,7-methylene group can be in α- or β position, pharmaceutical preparations containing at least one isomer of formula I and use thereof in the treatment of endometriosis.

The present invention relates to the object characterized in the patent claims, i.e. 18-methyl-6,7-methylene-3-oxo-17-pregn-4-ene-21,17β-carbolactones (formula I), wherein the methylene group in position 6,7 of the steroid skeleton can be in the α or β position, pharmaceutical preparations containing at least one of the stated isomers and use thereof in the treatment of endometriosis.

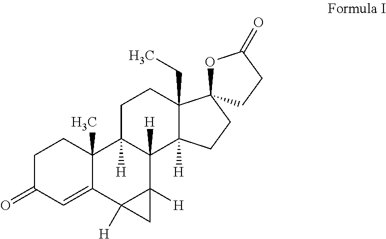

The problem to be solved by the present invention is to provide novel compounds for the treatment of endometriosis that display a better profile of action or side effects than currently available therapies. In particular, permanent or long-term treatment of endometriosis should be made possible by the compounds according to the invention.

Moreover, side effects such as occur when using gestagens for treating endometriosis, for example disturbances of bleeding, should be avoided by the new therapeutic approach.

The clinical picture of endometriosis has been investigated and described comprehensively, although the pathogenic mechanisms are still not known completely. Endometriosis is growth of endometrial tissue outside of the localization in the luminal region of the uterus. These so-called endometriotic lesions either occur in the muscular region of the uterus (endometriosis interna, adenomyosis) or at various sites in the abdominal cavity, e.g. the ligaments, on the parietal peritoneum of the Douglas pouch (peritoneal endometriosis), the intestinal wall, on the ovary (so-called endometrioma) or rectovaginally (rectovaginal, often also deeply infiltrating, endometriosis) and retain the properties of their original tissue.

In all its various manifestations, endometriosis is hormone-dependent and displays an essentially inflammatory character. It affects 10-20% of women of reproductive age. The disease only occurs in exceptional cases in women after the menopause. The core symptoms of endometriosis are chronic lower abdominal pain, dysmenorrhoea, dyspareunia, dysuria, bleeding disorders and infertility. The symptoms mostly occur in combination. It is presumed that the lesions enter the peritoneal space through so-called retrograde menstruation via the fallopian tube and then become implanted there.

Current therapeutic approaches for the treatment of diagnosed endometriosis are very limited.

Thus, endometriosis can be treated by surgical removal of the endometriotic lesions in a laparoscopic procedure. Endometriotic foci are removed surgically with heat (electrocauterizing) or by excision (extirpation). In addition, any adhesions present can be separated, endometriotic cysts removed and, if the patient wishes to have children, the patency of the fallopian tubes can be tested by chromopertubation. However, the relapse rate after such surgery is very high (25-30%). Hysterectomy, i.e. the complete removal of the uterus, is the final therapeutic option in these especially refractory cases.

In particularly severe diseases, sometimes only the removal of both ovaries and fallopian tubes (bilateral salpingo-oophorectomy, adnexectomy) provides definitive relief from symptoms.

The menstrual pains and prolonged or intensified bleeding that arise from endometriosis in the uterine musculature (adenomyosis uteri) can also be treated successfully with a hysterectomy.

However, these operations lead to infertility and early menopause with the associated problems, so that the benefits must be carefully weighed against the disadvantages.

In addition to invasive surgical procedures, consideration may also be given to drug therapy. This is often considered when large areas are affected, which possibly are not fully operable, but can also be applied in the case of mild to moderate disease. As well as pure pain therapy with non-steroidal anti-inflammatory drugs (NSAIDs), basically four groups of substances may be considered:
(a) combined oral contraceptives (OC, consisting of oestrogen and gestagen)
(b) gestagens
(c) GnRH analogues (GnRH=gonadotropin-releasing hormones) and
(d) Danazol®

Combined oral contraceptives (a) regulate the menstrual cycle and reduce the intensity of menstruation. This presumably accounts for their efficacy in endometriosis patients. However, it is assumed that on the one hand the relapse rate for pain symptoms is very high, and on the other hand new studies indicate that the use of these hormonal active substances over many years is associated with an increased rate of deeply infiltrating endometriosis¹, an especially painful form of endometriosis. ¹ Chapron et al. Hum Reprod. 2011 August; 26(8):2028-35

The use of OCs is also described in the patent literature. Thus, EP 1257280 discloses that micronized drospirenone is suitable for the treatment of endometriosis. It is described there, in paragraph [0045], that compositions of drospirenone with a low content of oestrogen or even without any oestrogen are suitable inter alia for the treatment of endometriosis. This is explained by the gestagenic property of drospirenone. Amounts from 0.5 to 10 mg of drospirenone are described as effective in EP1257280. This document does not disclose anything about the duration of treatment of endometriosis with drospirenone.

Mineralocorticoid receptor antagonists for the production of a medicinal product for treating endometriosis are described in WO2008/107373 A1. In addition to the use of compounds with pure antimineralocorticoid action, compounds are also proposed there that, additionally, also exert an action on the progesterone receptor, on the oestrogen receptor, on the glucocorticoid receptor and/or on the androgen receptor. In particular, the compounds spironolactone and the aforementioned drospirenone disclosed in WO2008/107373 A1 also have a gestagenic action.

The compound eplerenone mentioned in WO2008/107373 A1 as a pure MR antagonist displays a relatively weak in-vitro potency (cf. Table 1). MR antagonists are preferred which, in in-vitro transactivation assays, have an at least 10-times lower IC₅₀ compared with eplerenone.

In addition to drospirenone [0013], other gestagens (b) are also described in the treatment of endometriosis. This is based on the one hand on suppression of the function of the ovaries and on the other hand on bringing about terminal differentiation of the endometrium, decidualization, which ultimately leads to tissue necrosis.

Under the action of gestagens, the body “believes” a pregnancy has started and this creates an altered hormonal situation. Ovulation no longer takes place and the uterine mucosa regresses. As a rule the endometriosis symptoms then subside within 6 to 8 weeks.

Depot-MPA (medroxyprogesterone acetate) and Visanne© (dienogest) are approved for endometriosis treatment. In the case of MPA, owing to the anti-oestrogen action of the compound there may already be a decrease in bone mass after a duration of use of 6 months. Therefore it should never be used for a time longer than 2 years². During treatment with gestagens, moreover, common side effects are irregularities in the bleeding profile, breakthrough bleeding and breast tenderness³. ² Physician Information for depo-subQ provera 104; Subcutaneous depot medroxyprogesterone acetate versus leuprolide acetate in the treatment of endometriosis-associated pain; P. G. Crosignani et al., Human Reproduction Vol. 2 1, No. 1 pp. 248-256, 2006 ³ Brown et al., Cochrane Database Syst Rev. 2012 Mar. 14; 3:CD002122. McCormack Drugs. 2010 Nov. 12; 70(16):2073-88

Generally, in addition to the hormone cycle, gestagens also influence the bleeding profile, with bleeding disorders as a common side effect of gestagens. This also applies to substances that are active on the other hormone receptors and at the same time have a gestagenic activity, for example spironolactone. Through abnormal angiogenesis (new vessel formation, a process that occurs cyclically in the endometrium) during decidualization of the endometrium, the vessel walls become fragile and so-called breakthrough bleeding occurs, independently of menstrual bleeding, and is characteristic of chronic treatment with gestagens⁴. ⁴ Lockwood, Menopause. 2011 April; 18(4):408-11

Patients with endometriosis often also have so-called relative progesterone resistances⁵. It is assumed that progesterone signalling can be disturbed in the endometriotic lesions, and complete transformation and desquamation of the endometrium is blocked by progesterone resistance. Persistence of the lesions and chronic course of the disease can thus be promoted. Therapeutic approaches whose action is not dependent on progesterone signalling are required for permanent treatment of the disease. ⁵ Al-Sabbagh et al., Mol Cell Endocrinol. 2012 Jul. 25; 358(2):208-15

The gonadotropin-releasing hormone analogues (GnRH) (c) currently represent the gold standard of approved therapeutic agents against all stages of endometriosis. GnRH analogues block the pituitary gland completely. The menstrual cycle no longer occurs. These substances therefore temporarily shift the woman's body artificially into the menopause and therefore the endometriosis tissue also can no longer bleed. The tissue becomes hypotrophic.

Owing to the profile of side effects, this therapeutic approach is, however, also only suitable for short-term use (up to 6 months). Thus, GnRH-agonists induce postmenopausal symptoms, such as hot flushes (80-90%), sleep disorders (60-90%), dry vagina (30%), headaches (20-30%), mood changes (10%) and decrease in bone density with associated increased risk of osteoporosis.

Apart from the aforementioned side effects, on completion of treatment the normal cycle resumes within two to three months. In more than 60% of affected women, the symptoms of endometriosis then also return, so that a repeat treatment cycle must be considered.

Owing to the aforementioned disadvantages, GnRH analogues have not so far found wide application in the treatment of endometriosis, even though they displaced the standard therapy with Danazol®, a gestagenic androgen, that became established in the 1970s, owing to the somewhat better profile of side effects.

Danazol® (d) was the first “classical” agent for treatment of endometriosis and was the gold standard until the 1970s. In long-term use, Danazol® leads, like the male sex hormone testosterone, to masculinization of women. Further side effects are the known effects of androgens, such as acne, hyperandrogenism, hirsutism and (irreversible) change in pitch of the voice.

Danazol® acts, like the GnRH-agonists, on the pituitary gland, which is responsible for the production of hormones that stimulate the ovaries. As a result, the production of oestrogens in the ovaries is halted.

There is therefore an urgent need for alternative preparations, which permit non-invasive treatment of endometriosis and do not have the disadvantages of the prior art.

One problem to be solved by the present invention is therefore to provide novel substances that overcome the disadvantages of the prior art, in particular that avoid the side effects caused by gestagens, e.g. bleeding disorders, or the effects caused by oestrogen deficiency, such as loss of bone mass and depression, i.e. a problem to be solved by the invention is to provide non-gestagenic substances.

Another problem to be solved by the invention is to provide substances for chronic treatment, with an improved profile of side effects.

It was found that compounds of formula I

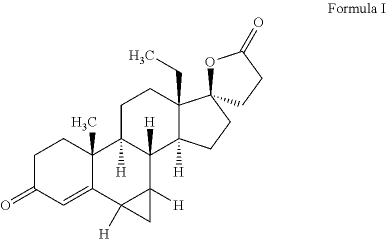

solve the aforementioned problems and are eminently suitable for use in the treatment of endometriosis. The 6β,7β-methylene isomer is especially preferred. The two isomers are mentioned for the first time in WO2012/059594 (FIGS. 4 and 5), which was filed on 4 Nov. 2012, and the present application claims the priority thereof.

The invention therefore relates to compounds of formula I, pharmaceutical preparations containing at least one isomer of formula I, and use thereof in the treatment of endometriosis.

Surprisingly, these compounds, both the 6β,7β isomer, which is described in more detail in example 5, Table 1, #1 (in-vitro transactivation data for mineralocorticoid and progesterone receptor, gestagenic in-vivo action), and the 6α,7α isomer, which is described in more detail in example 5, Table 1, #1A, do not display any gestagenic efficacy in relevant animal models, even though structurally very similar compounds, as disclosed for example by Muhn et al.⁶ or by Kuhnz et al.⁷, have gestagenic properties (see example 5 #2 & #3 in Table 1). In particular, the increase in the gestagenic in-vivo potency on inserting an additional methyl group in position 18 of the steroidal backbone, which is familiar to a person skilled in the art, surprisingly is not observed, as can be seen on comparing the entries in example 5 #1 and #2 vs. #3 and #4 in Table 1. ⁶ Muhn et al., Contraception 1994, 51:99-110 ⁷ Kuhnz et al., Contraception 1995, 51(2):131-139

The aforementioned compounds, and in particular the 6β,7β isomer, show a better profile of action and side effects than the treatments available until now and are therefore a better therapeutic agent against endometriosis.

Moreover, the compounds according to the invention, compared with the known mineralocorticoid receptor antagonists (eplerenone, spironolactone, drospirenone), are characterized by higher potency and absence of gestagenic action.

Compounds that have, in in-vitro transactivation assays, a 10-times lower IC₅₀ compared with eplerenone, are defined as potent mineralocorticoid receptor antagonists in the sense of the present invention.

Mineralocorticoid receptor antagonists without notable gestagenic activity are substances that do not display any action in in-vitro progesterone receptor transactivation assays and/or in in-vivo assays (gestagen-sensitive assays for maintenance of pregnancy).

The compound usable according to the invention is produced as described below. The synthesis route for the novel 18-methyl-6,7-methylene-3-oxo-17-pregn-4-ene-21,17β-carbolactones according to scheme 1 starts for example from Endion⁸ 2. ⁸ Kerb, Ger. Offen. (1970), DE 1921396, CAS [31320-40-8]

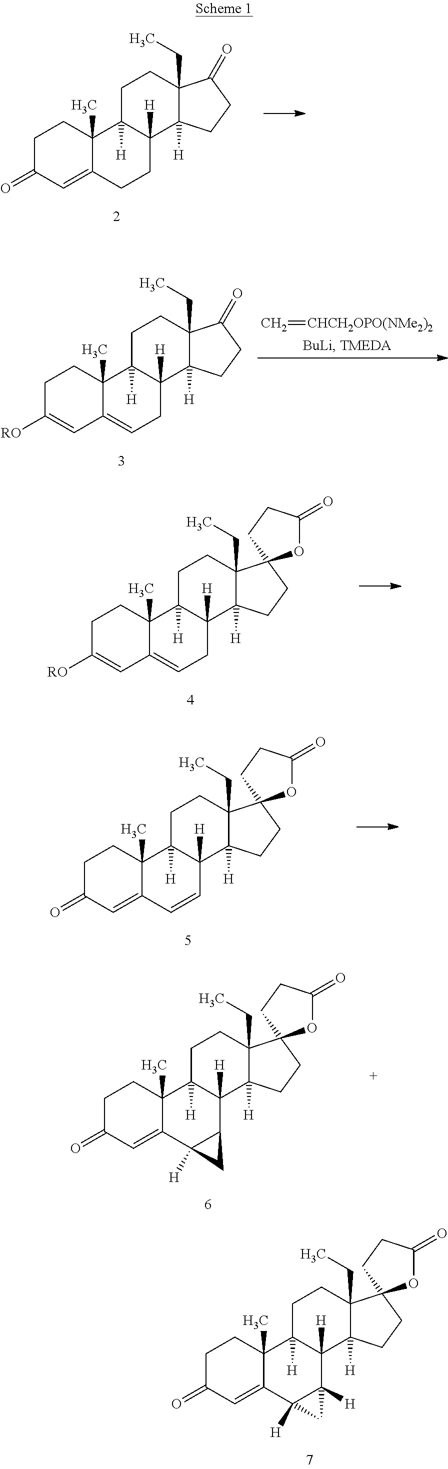

The dienol ether 3 is produced by isomerizing etherification of Endion 2 by known methods for example for R=-methyl with 2,2-dimethoxypropane and pyridinium-p-toluene sulphonate and the spirolactone 4 is established e.g. by the method of Sturtz⁹ or alternatively by known methods¹⁰. Introduction of the 6,7-double bond takes place via bromination of the 3,5-dienol ether 4 and subsequent cleavage of hydrogen bromide¹¹. ⁹ Sturz Synthesis 1980, 289 ¹⁰ Bittler Angew. i.e. 21 1982, 696; Laurent J. Steroid Biochem. 19 1983, 771 ¹¹ J. Fried, J. A. Edwards, Organic Reactions in Steroid Chemistry, von Nostrand Reinhold Company 1972, S. 265-374)

The dienol-ether bromination can be carried out for example similarly to the specification of J. A. Zderic, et al.¹². The hydrogen bromide is split off by heating the 6-bromo compound with basic reagents, for example lithium bromide or lithium carbonate in aprotic solvents such as dimethylformamide at temperatures of 50-120° C. or by heating the 6-bromo compounds in a solvent such as collidine or lutidine to compound 5¹³. This is then converted by cyclopropanation of the 6,7-double bond by known methods, e.g. with dimethylsulphoxonium methylide [see e.g. DE-A 11 83 500, DE-A 29 22 500, EP-A 0 019 690, U.S. Pat. No. 4,291,029; E. J. Corey and M. Chaykovsky, J. Am. Chem. Soc. 84, 867 (1962)] to the compounds of formula I according to the invention, i.e. the stereoisomers of formula 6 and 7. The mixture of the 6,7-α- and β-stereoisomers can be separated e.g. by chromatography into the individual isomers. ¹² J. A. Zderic, Humberto Carpio, A. Bowers and Carl Djerassi in Steroids 1,233 (1963) ¹³ Strike in FR 1529949 (1968), CAS[23675-27-6]

The active substance or active substances can be mixed with the usual excipients. The mineralocorticoid receptor antagonists are formulated in a manner known per se by a person skilled in the art.

The therapeutically effective dose depends on body weight, route of application, individual response, the type of preparation and the time point or interval when application takes place. A typical dose range for a woman with 70 kg body weight is between 1 and 100 mg/day, preferably between 5 and 20 mg/day. A dose of 10 mg/day is especially preferred.

The present invention further relates to medicinal products containing at least one compound according to the invention and optionally at least one or more other active substances, and use thereof for the treatment of endometriosis. As suitable combination active substances, we may mention for example and preferably: selective oestrogen receptor modulators (SERMs), oestrogen receptor (ER) antagonists, aromatase inhibitors, 17β-HSD1 inhibitors, steroid sulphatase (STS) inhibitors, suitable GnRH-agonists (especially super-agonists) and antagonists, kisspeptin receptor (KISSR) antagonists, selective androgen receptor modulators (SARMs), 5α-reductase inhibitors, selective progesterone receptor modulators (SPRMs), gestagens, antigestagens, oral contraceptives, inhibitors of mitogen activated protein (MAP) kinases and inhibitors of MAP kinase kinases (Mkk3/6, Mek1/2, Erk1/2), inhibitors of protein kinases B (PKBα/β/γ; Akt1/2/3), inhibitors of phosphoinositide-3-kinases (PI3K), inhibitors of cyclin-dependent kinase (CDK1/2), inhibitors of the hypoxia induced signalling pathway (HIF1alpha inhibitors, activators of prolylhydroxylases), histone deacetylase (HDAC) inhibitors, prostaglandin F receptor (FP) (PTGFR) antagonists or non-steroidal anti-inflammatory drugs (NSAIDs).

The compounds according to the invention can have systemic and/or local action. For this purpose, they can be applied by a suitable route, e.g. oral, parenteral, pulmonary, nasal, sublingual, lingual, buccal, rectal, dermal, transdermal, conjunctival, topical or as implant.

For these routes of application, the compounds to be used according to the invention can be transformed into suitable application forms.

If further active substances are present in addition to the compound of the invention according to formula I, these can be formulated in a common application form or optionally can also be administered as a combination preparation.

Dosage forms that contain the compounds according to the invention in crystalline and/or amorphized and/or dissolved form, e.g. tablets (uncoated or coated tablets, for example with enteric coatings or slow-dissolving or insoluble coatings, which control the release of the compound to be used according to the invention), tablets or films/wafers that disintegrate rapidly in the oral cavity, films/lyophilizates, capsules (for example hard or soft gelatin capsules), sugar-coated pills, granules, pellets, powders, emulsions, suspensions, aerosols or solutions functioning according to the prior art, with rapid and/or modified release of the compounds to be used according to the invention, are suitable for oral application.

Parenteral application can take place with avoidance of an absorption step (e.g. intravenous, intra-arterial, intracardiac, intraspinal or intralumbar application) or including absorption (e.g. intramuscular, subcutaneous, intracutaneous, percutaneous or intraperitoneal application). Among others, preparations for injection and infusion in the form of solutions, suspensions, emulsions, lyophilizates or sterile powders are suitable as dosage forms for parenteral application.

For example pharmaceutical forms for inhalation (inter alia powder inhalers, nebulizers), nasal drops, solutions or sprays, tablets, films/wafers or capsules for lingual, sublingual or buccal application, suppositories, vaginal capsules, aqueous suspensions (lotions, shaking mixtures), lipophilic suspensions, ointments, creams, transdermal therapeutic systems (e.g. patches), milk, pastes, foams, dusting powders or implants are suitable for the other routes of application.

Oral or parenteral application, especially oral and intravenous application, are preferred.

The compounds to be used according to the invention can be transformed to the aforementioned dosage forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. oxidants, for example ascorbic acid), colorants (e.g. inorganic pigments, for example iron oxides) and taste and/or odour correctants.

Nevertheless, it may optionally be necessary to deviate from the stated amounts, namely depending on body weight, route of application, individual response to the active substance, type of preparation and time point or interval when application takes place. Thus, in some cases it may be sufficient to use less than the aforementioned minimum amount, whereas in other cases the stated upper limit must be exceeded. For administration of larger amounts, it may be advisable to distribute these in several individual doses throughout the day.

As was found in the endometriosis animal model (mouse example 4), when the compound according to the invention is used in the stated dose range, a reduction in size of the endometrial lesions can be observed in vivo.

Surprisingly, despite high in-vitro potency on the MR, the compounds according to the invention do not display any gestagenic effects (cf. Table 1, example 5).

### EXAMPLES

The invention is explained by means of the following examples, without these examples being in any way limiting.

### Example 1

### 18-Methyl-3-oxo-17-pregna-4,6-diene-21,17β-carbolactone

### a) 3-Methoxy-18-methyl-androsta-3,5-dien-17-one

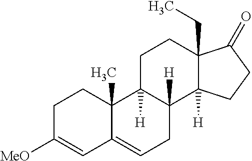

3.9 g of pyridinium tosylate was added to a solution of 27 g of 18-methyl-androsta-4,6-diene-3,17-dione (Kerb, Ger. Offen. (1970), DE 1921396, CAS [31320-40-8]) in 540 ml of 2,2-dimethoxypropane. Then it was stirred for 8 h at 100° C. bath temperature. After cooling to room temperature, 5.3 ml of pyridine was added and it was evaporated to dryness under vacuum. The residue was precipitated with 80 ml methanol and filtered with suction. 24.8 g of 3-methoxy-18-methyl-androsta-3,5-dien-17-one was obtained as a colourless solid.

¹H-NMR (400 MHz, CDCl₃): δ=5.29-5.22 (m, 1H), 5.14 (s, 1H), 3.58 (s, 3H), 2.43 (dd, 1H), 2.36-2.20 (m, 2H), 2.16-2.03 (m, 3H), 1.99-1.57 (m, 8H), 1.49-1.04 (m, 7H), 1.00 (s, 3H), 0.80 (t, 3H) [ppm].

### b) 3-Methoxy-18-methyl-17-pregna-3,5-diene-21,17β-carbolactone

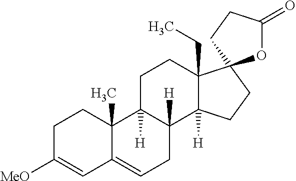

36.5 g of allyl-tetramethylphosphorodiamidate, dissolved in 59 ml tetrahydrofuran, was added dropwise to 226 ml of 1.6M butyllithium solution (in hexane) at −55° C. After stirring for 1 h at −55° C., it was heated to −20° C., 46 ml of N,N,N,N-tetramethylethanediamine was added and it was allowed to warm to room temperature. A solution of 18.9 g of 3-methoxy-18-methyl-androsta-3,5-dien-17-one in 213 ml tetrahydrofuran was added and it was stirred for a further 5 hours at 80° C. Then saturated aqueous ammonium chloride solution was added and it was poured into water, extracted three times with ethyl acetate, washed with water and sodium chloride solution until neutral, dried over sodium to sulphate, and evaporated under vacuum at 40° C. 22.3 g of 3-methoxy-18-methyl-17-pregna-3,5-diene-21,17β-carbolactone was obtained.

¹H-NMR (400 MHz, chloroform-d): δ=5.26-5.20 (m, 1H), 5.13 (s, 1H), 3.58 (s, 3H), 2.54-2.25 (m, 5H), 2.19 (dt, 1H), 2.10 (dd, 1H), 1.97-1.77 (m, 5H), 1.73-1.51 (m, 6H), 1.42 (qd, 1H), 1.34-1.12 (m, 4H), 0.98 (s, 3H), 0.97 (t, 3H) [ppm].

### c) 1 8-Methyl-3-oxo-1 7-pregna-4,6-diene-21,17β-carbolactone

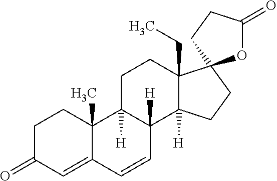

22 ml of a 10% sodium acetate solution and then 8.84 g of 1,3-dibromo-5,5-dimethylhydantoin in portions were added to a suspension of 22 g of 3-methoxy-18-methyl-17-pregna-3,5-diene-21,17β-carbolactone in 220 ml dimethylformamide at 0° C., it was stirred for 0.5 h at 0° C. (ice bath), 8.25 g lithium bromide and 7.24 g lithium carbonate were added, and it was stirred for 5 hours at 80° C. bath temperature. Then it was added with stirring to ice water/common salt, the precipitate was filtered, washed with water, and chromatographed on silica gel with hexane/ethyl acetate. 10.7 g of 18-methyl-3-oxo-17-pregna-4,6-diene-21,17β-carbolactone was obtained.

¹H-NMR (400 MHz, chloroform-d): δ=6.17-6.12 (m, 1H), 6.12-6.07 (m, 1H), 5.69 (s, 1H), 2.66-2.32 (m, 7H), 2.07-1.79 (m, 5H), 1.78-1.50 (m, 6H), 1.46-1.36 (m, 1H), 1.32-1.17 (m, 3H), 1.13 (s, 3H), 1.01 (t, 3H) [ppm].

### Example 21

### 8-Methyl-6β,7β-methylene-3-oxo-17-pregn-4-ene-21,17β-carbolactone (6)

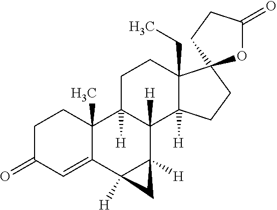

A suspension of 50 g of trimethylsulphoxonium iodide in 750 ml dimethylsulphoxide with 9.73 g sodium hydride (55% in oil) was dissolved for 2 hours at room temperature under argon, 24.7 g of 18-methyl-3-oxo-17-pregna-4,6-diene-21,17β-carbolactone (prepared as described in example 1) was added and it was stirred for a further 24 hours at room temperature. Then it was added with stirring to 15 l ice water/common salt, the precipitate was filtered, washed with water and dried under vacuum at 60° C. 22.4 g of crude product was obtained. According to silica gel chromatography with hexane/ethyl acetate as fraction II, 7.5 g of 18-methyl-6β,7β-methylene-3-oxo-17-pregn-4-ene-21,17β-carbolactone. 210-211° C., [α]_{D}−151.9°+/−0.05° (chloroform, c=10 mg/ml)

¹H-NMR (600 MHz, chloroform-d): δ=6.02 (s, 1H), 2.62-2.44 (m, 3H), 2.43-2.36 (m, 3H), 1.98-1.81 (m, 6H), 1.75-1.65 (m, 2H), 1.65-1.42 (m, 8H), 1.28-1.11 (m, 4H), 1.09 (s, 3H), 1.08-1.01 (m, 2H), 0.96 (t, 3H), 0.80 (q, 1H) [ppm].

### Example 3

### 18-Methyl-6α,7α-methylene-3-oxo-17-pregn-4-ene-21,1713-carbolactone (7)

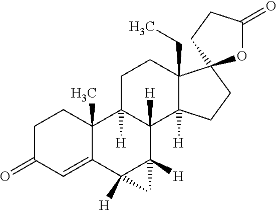

According to the method in example 2, after chromatography as fraction I, 2.9 g of 18-methyl-6α,7α-methylene-3-oxo-17-pregn-4-ene-21,17β-carbolactone was obtained as a solid with melting point of 230-231° C., [α]_{D}=102.6°+/−0.11° (chloroform, c=10 mg/ml).

¹H-NMR (600 MHz, chloroform-d): δ=5.95 (s, 1H), 2.58-2.44 (m, 3H), 2.43-2.34 (m, 3H), 2.21-2.15 (m, 2H), 1.98-1.86 (m, 5H), 1.81-1.74 (m, 2H), 1.65-1.48 (m, 6H), 1.34-1.26 (m, 2H), 1.17-1.07 (m, 2H), 1.14 (s, 3H), 0.98 (t, 3H), 0.89-0.84 (m, 1H), 0.77-0.71 (m, 1H), 0.47 (q, 1H) [ppm].

### Example 4

### Endometriosis Models In Vivo

Xenograft Endometriosis Model with Primate Endometrium:

To investigate the effect of the compound according to the invention on the growth of endometriotic lesions, a xenograft endometriosis model was used in immunodeficient SCID mice, in which endometrium from rhesus macaques was implanted.

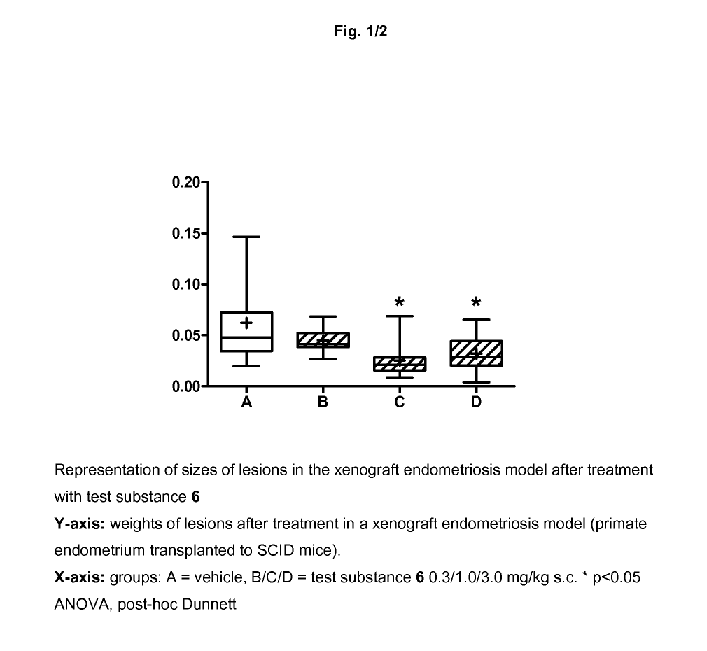

The ovariectomized SCID mice were given estradiol and progesterone capsules as hormone replacement, in order to create an optimum hormonal environment for the primate endometrium. Donor monkeys were treated with estradiol and progesterone for 7 days. Then the endometrium of the animals was curetted and cut into 2×2×4 mm pieces. The endometrium was transplanted into the abdominal cavity of the mice by laparotomy or was implanted subcutaneously. The lesions were allowed to grow for 14 days with estradiol and progesterone treatment, followed by 14 days of estradiol treatment (corresponding to one menstrual cycle). The treatment started with daily s.c. administration of the compound according to the invention for a period of 28 days with dosages of 0.3, 1.0 and 3.0 mg/kg, and estradiol supplementation was continued. After the treatment time, the animals underwent a final laparotomy and the weight of the lesions per animal was determined. Spironolactone was used as positive control at a dose of 10 mg/kg (vehicle: benzyl benzoate/castor oil). The compound according to the invention 6 showed, at 1.0 and 3.0 mg/kg/d, a significant effect on growth of the lesion compared to the vehicle (A) or the lowest dose group (B=0.3 mg/kg). The result of the measurements is shown in FIG. 1/2.

### Syngeneic Mouse Endometriosis Model:

The syngeneic induction of endometriosis in mice is a commonly used animal model for testing the efficacy of substances for treating endometriosis. Endometriosis is induced experimentally by transplanting murine uterus fragments from a donor mouse of the same strain into the abdominal cavity of the recipient mouse. Female animals of the balb/c strain were used. The cycle of the mouse was determined by vaginal smear. Donor animals that were in oestrus were used exclusively. The donor animals were killed and the uterine horns were removed and then cut open longitudinally. Using a punch, 2 mm biopsy specimens were punched out of the uteri, and were then sutured into the recipient animal. The recipient animals were anaesthetized and submitted to a laparotomy. During the operation, 6 uterus punch samples from a donor mouse were sutured onto the parietal peritoneum of the recipient mouse. On the day after this surgery, 4-week treatment with the test substances was begun (vehicle: Tween80/Captex 200P). After 28 days, the animals were opened in a final laparotomy and the sizes of the lesion were determined. The enlarged lesions were recorded photographically and the area was measured using AxioVision software. 14 animals were used per treatment group.

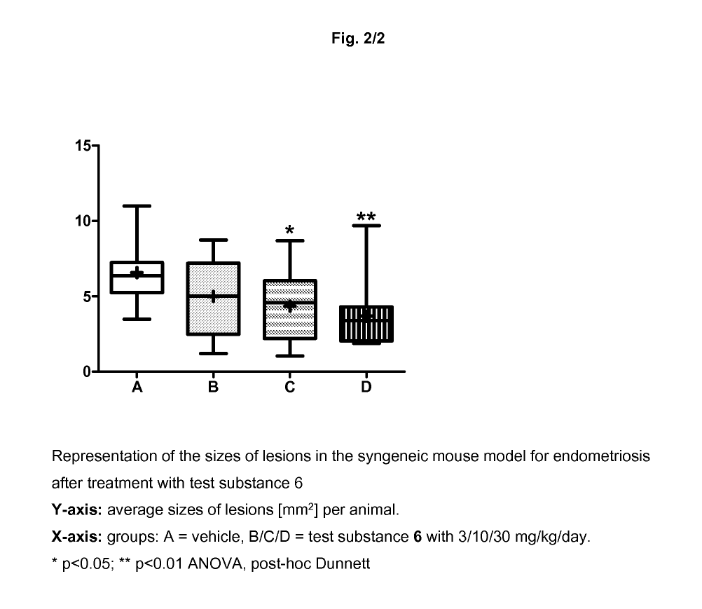

In this case, the test substance 6 was tested in 3 different dosage schemes and the size of the lesion was evaluated compared with the animals treated with the vehicle (group A). The following dosages were tested: 3, 10 and 30 mg/kg/day (groups B, C, D). FIG. 2/2 shows the average sizes of the lesions (in mm²) per animal (y-axis).

### Example 5

### In-Vitro/In-Vivo Action on MR and PR

Table 1 shows the in-vivo data for the gestagenicity of the substances. The gestagenicity in vivo can be determined by two different assays, on the one hand by the pregnancy maintenance test in the rat, on the other hand by the McPhail test in the rabbit (endometrial transformation). The available data for the compounds according to the invention 6 and 7 are shown (Table 1 #1 and #1A), and for spironolactone for comparison. To ensure comparability among the assays, the gestagens drospirenone and levonorgestrel are shown, for which data are known from both assays. Furthermore, norethisterone is shown, to illustrate, together with levonorgestrel, the effect of an 18-methyl group on gestagenic potency.

TABLE 1

| | Structure of the | Designation of | | ED₅₀ | |
|---|---|---|---|---|---|
| # | compound | the compound | IC₅₀ (MR) | (PR) | in vivo PR |
| #1 | 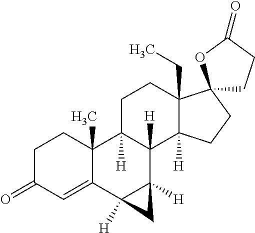 | 18-Methyl-6β,7β- methylene-3-oxo-17- pregn-4-ene-21,17β- carbolactone | 0.71 nM | 1.2 nM (50% efficacy) | no effect (highest dosage tested: 30 mg/kg/d s.c., pregnancy maintenance in the rat) |
| #1A | 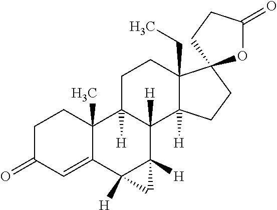 | 18-Methyl-6α,7α- methylene-3-oxo-17- pregn-4-ene-21,17β- carbolactone | 5.6 nM | 100 nM (38.6% efficacy) | no effect (highest dosage tested: 30 mg/kg/d s.c., pregnancy maintenance in the rat) |

#2-6 = Comparative examples

| | | | | | |
|---|---|---|---|---|---|
| #2 | 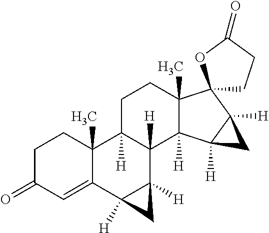 | Drospirenone (DRSP) | 1.48 nM | 3.6 nM (83% efficacy) | Pregnancy maintenance: 12 mg/kg s.c. (80%) ⁶ McPhail test in the rabbit: EDmin ~0.2 mg/kg/d ⁶ |
| #3 | 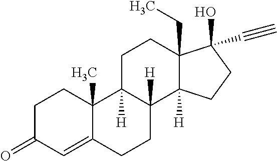 | Levonorgestrel (LNG) | 337 nM | 1.6 nM (108% efficacy) | pregnancy maintenance: 1.2 mg/kg s.c. (80%) ⁷ McPhail test in the rabbit: EDmin ~0.02 mg/kg/d ⁶ |
| #4 | 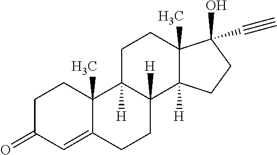 | Norethisterone | not investigated | 4.1 nM (87.9% efficacy) | McPhail test in the rabbit: EDmin ~0.3 mg/kg/d ¹⁴ |
| #5 | 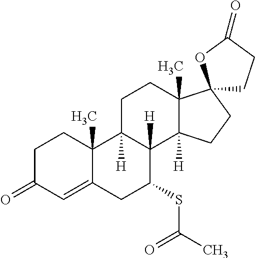 | Spironolactone | 10.5 nM | 666 nM (43% efficacy) | McPhail test in the rabbit: EDmin ~15 mg/kg/d s.c. ⁶ |
| #6 | 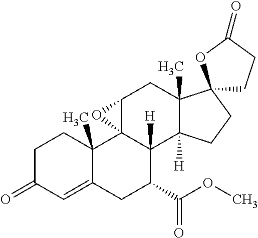 | Eplerenone | 1.10 μM | >1 μM | not investigated |

⁶ Muhnet al., Contraception 1994, 51: 99-110
⁷ Kuhnz el al., Contraception 1995, 51(2): 131-139
¹⁴ Phillips et al., Contraception 1987, 36(2): 181-192

The in-vitro transactivation data (IC₅₀ values) and the gestagenic in-vivo activity were determined as described below.

### 1. Cellular In-Vitro Test for Determining the Inhibitory MR Activity and MR Selectivity Versus Other Steroid Hormone Receptors

A recombinant cell line is used for identifying antagonists of the human mineralocorticoid receptor (MR) and for quantifying the efficacy of the compounds described here. The cell was originally derived from an ovarian epithelial cell of the hamster (Chinese Hamster Ovary, CHO K1, ATCC: American Type Culture Collection, VA 20108, USA). In this CHO K1 cell line, an established chimeric system is used, in which the ligand-binding domains of human steroid hormone receptors are fused onto the DNA-binding domain of the yeast transcription factor GAL4. The resultant GAL4 steroid hormone receptor chimeras are co-transfected in the CHO cells with a reporter construct and stably expressed.

### Cloning:

For generating the GAL4 steroid hormone receptor chimeras, the GAL4-DNA-binding domain (amino acids 1-147) from the vector pFC2-dbd (from the company Stratagene) with the PCR-amplified ligand-binding domains of the mineralocorticoid receptor (MR, amino acids 734-985) and of the progesterone receptor (PR, amino acids 680-933) cloned into the vector pIRES2 (from the company Clontech). The reporter construct, which contains five copies of the GAL4 binding site, upstream of a thymidine kinase promoter, leads to expression of firefly luciferase (Photinus pyralis) after activation and binding of the GAL4 steroid hormone receptor chimeras by the respective specific agonists aldosterone (MR) and progesterone (PR).

### Test Procedure:

The MR and PR cells are plated out on the day before the test in medium (Optimem, 2.5% FCS, 2 mM glutamine, 10 mM HEPES) in 96-well (or 384-well or 1536-well) microtitre plates and kept in a cell incubator (96% air humidity, 5% v/v CO₂, 37° C.). On the day of the test, the test substances are taken up in the aforementioned medium and are added to the cells. About 10 to 30 minutes after adding the test substances, the respective specific agonists of the steroid hormone receptors are added. After a further incubation time of 5 to 6 hours, the luciferase activity is measured by means of a video camera. The measured relative light units yield, as a function of the concentration of substance, a sigmoidal stimulation curve. The IC₅₀ values are calculated using the computer program GraphPad PRISM (version 3.02).

### 2. Test for Gestagenic Activity In Vivo: Pregnancy Maintenance in the Rat

The pregnancy maintenance test is a model in which the response of the endometrium to a gestagen is investigated very sensitively. A pregnancy is only continued in the presence of an effective gestagen. Pregnant rats are oophorectomized and are treated with test substance or positive control for a period of 7 days. At the end of the treatment, the number of living and dead fetuses is determined as a measure of the gestagenic, i.e. pregnancy-maintaining action, of the test substance.

### 3. Test for Gestagenic Activity In Vivo: McPhail Assay in the Rabbit

Female rabbits are ovariectomized. 7 days after ovariectomy, the animals are given estradiol for 6 days. The animals are treated with the test substance for 5 days, and then the uterus is removed and prepared histologically. The secretory transformation of the endometrium is evaluated as a measure of the gestagenic action (threshold dose, at which there is onset of a secretory transformation).

1. 18-Methyl-6,7-methylene-3-oxo-17-pregn-4-ene-21,17β-carbolactones of formula I, in which the 6,7-methylene group can be in the α or β position

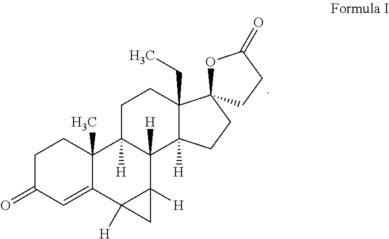

2. Compound according to claim 1, namely 18-methyl-6β,7β-methylene-3-oxo-17-pregn-4-ene-21,17β-carbolactone.

3. 18-Methyl-6,7-methylene-3-oxo-17-pregn-4-ene-21,17β-carbolactone for use in the treatment of endometriosis.

4. 18-Methyl-6β,7β-methylene-3-oxo-17-pregn-4-ene-21,17β-carbolactone for use in the treatment of endometriosis.

5. Pharmaceutical preparations containing at least one compound according to claim 1 and at least one pharmaceutically harmless carrier.

6. Pharmaceutical preparations according to claim 5 further comprising at least one other active pharmaceutical ingredient selected from the group of selective oestrogen receptor modulators (SERMs), oestrogen receptor (ER) antagonists, aromatase inhibitors, 17β-HSD1 inhibitors, steroid sulphatase (STS) inhibitors, GnRH agonists and antagonists, kisspeptin receptor (KISSR) antagonists, selective androgen receptor modulators (SARMs), 5α-reductase inhibitors, selective progesterone receptor modulators (SPRMs), gestagens, antigestagens, oral contraceptives, inhibitors of mitogen activated protein (MAP) kinases and inhibitors of MAP kinase kinases (Mkk3/6, Mek1/2, Erk1/2), inhibitors of protein kinases B (PKBα/β/γ; Akt1/2/3), inhibitors of phosphoinositide-3-kinases (PI3K), inhibitors of cyclin-dependent kinase (CDK1/2), inhibitors of the hypoxia-induced signalling pathway (HIF1alpha inhibitors, activators of prolylhydroxylases), histone deacetylase (HDAC) inhibitors, prostaglandin F receptor (FP) (PTGFR) antagonists or non-steroidal anti-inflammatory drugs (NSAIDs) in a pharmaceutically harmless carrier.

7. Pharmaceutical preparations according to claim 6 containing a compound according to claim 1 and at least one other active pharmaceutical ingredient selected from the group of ER antagonists, aromatase inhibitors, kinase inhibitors or NSAIDs.
